(19) United States
(12) Patent Application Publication
(10) Pub. No.: US 2004/0243146 A1
(43) Pub. Date: Dec. 2, 2004

(54) Method and apparatus for supporting a medical device

(76) Inventors: Richard M. Chesbrough, Bloomfield Hills, MI (US); John Brown, Harwinton, CT (US); Mark Broyles, Plainville, CT (US)

Correspondence Address: McCarter & English LLP, CityPlace I, 185 Asylum Street, Hartford, CT, 06103-3495, US

(21) Appl. No.: 10/715,587

(22) Filed: Nov. 18, 2003

Related U.S. Application Data
(60) Provisional application No. 60/427,025, filed on Nov. 18, 2002.

(51) Int. Cl. A61B 19/00

(52) U.S. Cl. 606/130; 604/385.06

(57) ABSTRACT

Methods and apparatus for use in supporting and/or guiding a medical device to be inserted into the body of a patient.

### CROSS REFERENCE TO RELATED APPLICATIONS

This application claims priority to U.S. Provisional Patent Application Ser. No. 60/427,025, filed Nov. 18, 2002, entitled “JOYSTICK: BIOPSY GUIDANCE SYSTEM”, which is incorporated by reference herein.

### FIELD OF THE INVENTION

The present invention relates to methods and apparatus for use in supporting and/or guiding a medical device to be inserted into the body of a patient.

### RELATED INFORMATION

Many surgical procedures call for a device to be inserted into the body of a patient. Examples of such devices include but are not limited to biopsy needles and catheters. It is generally desirable to be able to insert such devices at a particular location on the body and/or at a particular angle relative to the body in order to collect a tissue sample and/or drain fluid from a particular position within the body. However, many of these devices do not remain in the desired orientation unless adequately supported. Support is sometimes provided using a combination of towels, pillows and sheets, or by hand. Because these types of support are somewhat inconvenient to employ, other methods for supporting such devices have been developed. Some of such methods employ a support that surrounds the device to be inserted. These supports are often retained to a base that is removably attached to the body of the patient. Notwithstanding the usefulness of these types of supports, there are occasions where it would be desirable to be able to remove the support without the need to remove the device from the patient's body. Unfortunately, many of such supports cannot be removed until after the device has been removed, thereby rendering such methods unusable in such occasions.

Thus, it would be desirable to provide a support that can be separated from such a device without the need to remove the device from the patient's body.

There are also occasions where it would desirable to be able to remove the support used in the course of one procedure in order substitute another type of support for a second procedure. For example, in order to drain fluid from a patient's body, a needle is often inserted first and a catheter inserted thereafter. Because the needle and catheter may warrant different types of supports, it would be desirable to remove the support used to support the needle and substitute a support adapted to support the catheter. Although it might be possible to accomplish this by swapping out the entire the support mechanism, such a method would unfortunately require removal and replacement of the portion attached to the patient's body.

Thus, it would be desirable to provide a support that can be replaced without the need to remove the base that connects the support to the body of the patient.

Additional methods and apparatus for retaining a support to a base are also desired.

Methods and apparatus to aid in selecting the proper orientation of a device are also desired. For example, every day, in hospitals and clinics around the country, thousands of patients undergo ultrasound and CT guided biopsies and injections of lesions and fluid collections in the chest, abdomen, pelvis and/or other areas in the body. Often the localization of these lesions is quite time consuming and may take longer than the actual biopsy, injection or drainage procedure. Many times the physician places the biopsy needle at the wrong angle to the intended target, resulting in misplacement and improper angulation of the device. This can cause excessive pain and bleeding, and increases the risk of injury to underlying organs. In addition, there is often a long period of “trial and error” until the needle is finally angled correctly to the lesion, and can be advanced to the appropriate level. This guesswork means wasted time, increased costs due to excess room time, and potentially increased radiation dose to the patient.

### SUMMARY OF THE INVENTION

According to a first aspect of the present invention, an apparatus for use in supporting a device to be introduced into a body of a patient includes: a base; a guide member that defines a channel for receiving the device to be introduced into the body of a patient; and a guide support being removably connected to the base and releasably capturing the guide member; wherein the guide support has at least one state in which the guide support defines a first opening, the guide member has at least one state in which the guide member defines a second opening, each of said openings having a width that is at least as wide as the channel to permit the guide support and the guide member to be separated from the device without removing the device from the body of the patient.

One advantage of a currently preferred embodiment of the above aspect of the present invention is that the guide member and the guide support may each be separated from a device that is inserted into a patient without the need to remove the device from the patient.

According to another aspect of the present invention, an apparatus for use in supporting a device to be introduced into a body of a patient includes: a base having a surface with adhesive adhered thereto for removably adhering the base to the body of a patient; a guide member that defines a channel for receiving the device to be introduced into the body of the patient; and a guide support that is removably connected to the base and releasably captures the guide member; wherein the guide support has at least one state in which the guide support defines a first opening and the guide member has at least one state in which the guide member defines a second opening to permit the guide member to be separated from the device without removing the device from the body of the patient.

A currently preferred embodiment of the above aspect of the present invention also provides the above mentioned advantage, i.e., that the guide member and the guide support may each be separated from a device that is inserted into a patient without the need to remove the device from the patient.

According to another aspect of the present invention, an apparatus for use in supporting a device to be introduced into a body of a patient includes: a base defining a mount and having a surface with adhesive adhered thereto for removably adhering the base to the body of a patient; a first guide releasably mountable to the mount and defining a first channel having a first configuration; and a second guide releasably mountable to the mount and defining a second channel having a second configuration that is different than the first configuration.

One advantage of a currently preferred embodiment of the above aspect of the present invention is that a single base can accommodate different types of guide members, thereby making it possible to remove the support used in the course of a procedure in order substitute another type of support, without the need to remove the base that connects the support to the body of the patient.

According to another aspect of the present invention, an apparatus for use in supporting a device to be introduced into a body of a patient includes: a base; a guide member that defines a channel for receiving an device to be introduced into a body of a patient; and a guide support including two portions pivotably connected to one another, a first positioning of the two portions defining a first state of the guide support, a second positioning of the two portions defining a second state of the guide support, the guide support further including a catch that is engaged with the base to releasably mount the guide support thereto when the guide support is in the first state and is disengaged from the base when the guide support is in the second state.

One advantage of a currently preferred embodiment of the above aspect of the present invention is that the guide support can be disengaged from the base by adjusting the position of the pivotably connected portions of the guide support.

According to another aspect of the present invention, an apparatus for use in supporting a device to be introduced into a body of a patient includes: a base; a guide member that defines a channel for receiving the device to be introduced into the body of a patient; and a guide support including two portions pivotably connected to one another, a first positioning of the two portions defining a first state of the guide support, a second positioning of the two portions defining a second state of the guide support, wherein the guide member is releasably captured in the guide support when the guide support is in the second state.

One advantage of a currently preferred embodiment of the above aspect of the present invention is that the guide member is releasably captured in the guide support by adjusting the position of the pivotably connected portions thereof.

According to another aspect of the present invention, an apparatus for use in supporting a device to be introduced into a body of a patient includes: a base for releasable attachment to the body of the patient; a guide member defining a channel for receiving the device to be introduced into the body of a patient; and a guide support including two portions movable relative to one another, a first positioning of the two portions defining a first state of the guide support, a second positioning of the two portions defining a second state of the guide support, wherein the guide member is releasably captured in the guide support when the guide support is in the first state and at least one of the guide support portions may be moved in a direction generally parallel to a major outer surface of the base plate to release the guide.

One advantage of a currently preferred embodiment of the above aspect of the present invention is that the guide member can be separated from a device that is inserted into a patient without the need to remove the device from the patient.

According to another aspect of the present invention, an apparatus for use in supporting a device to be introduced into a body of a patient includes: a substantially flat, flexible base; and a guide member that defines a channel for receiving the device to be introduced into the body of a patient and is disposed in register with a marginal portion of the base; wherein the guide member has at least one state in which the guide support defines an opening to permit the guide member to be separated from the device without removing the device from the body of the patient.

Further aspects of the present invention include the methods disclosed herein, alone or in combination, for use in supporting a device to be introduced into a body of a patient.

One advantage of a currently preferred embodiment of the above aspect of the present invention is that the guide member is releasably captured in the guide support by adjusting the position of the portions of the guide support.

Other advantages of the above described aspects of the present invention will become apparent in view of the following detailed description of preferred embodiments, claims, and accompanying drawings.

It should be understood, however, that unless otherwise specified, the advantages noted herein are not requirements of the present invention.

### BRIEF DESCRIPTION OF THE DRAWINGS

FIG. 1 is a perspective view of a first embodiment of an apparatus for supporting a medical device;

FIG. 2 is cross sectional view of the apparatus of FIG. 1 taken along the line 2-2;

FIG. 3 is a perspective view of a second embodiment of an apparatus for supporting a medical device, shown in a disassembled state;

FIG. 4 is a top elevational view of the guide member of the apparatus of FIG. 3;

FIG. 5 is a top elevational view of a third embodiment of an apparatus for supporting a medical device;

FIG. 6 is a perspective view of a fourth embodiment of an apparatus for supporting a medical device;

FIG. 7 is an enlarged, side elevational view of a portion of the apparatus of FIG. 6;

FIG. 8 is a perspective view of a first embodiment of an apparatus for supporting a medical device;

FIG. 9 is a perspective view of the apparatus and medical device of FIG. 8, prior to inserting the medical device into the apparatus;

FIG. 10 is a top elevational view of the apparatus of FIG. 8;

FIG. 11 is a side elevational view of the apparatus of FIG. 8;

FIG. 12 is a side elevational view of the apparatus of FIG. 8;

FIG. 13 is a cross section view of the apparatus of FIG. 8 taken along the line 13-13 shown in FIG. 10;

FIG. 14 is a perspective view of the apparatus of FIG. 8 with the guide support in an open state;

FIG. 15 is a perspective view of the apparatus of FIG. 8 with the guide support disassembled from the apparatus and in an open state;

FIG. 16 is a perspective view of the apparatus of FIG. 8 with the guide member and guide support disassembled from the apparatus and in an open state;

FIG. 17 is a perspective view of the base of the apparatus of FIG. 8;

FIG. 18 is a perspective view of a second embodiment of the guide member of the apparatus of FIG. 8;

FIG. 19 is a perspective view of a third embodiment of the guide member;

FIG. 20 is a perspective view of a fourth embodiment of the guide member;

FIG. 21 is a perspective view of a second embodiment of an apparatus for supporting a medical device, with the guide member disassembled from the base and in an open state;

FIG. 22 is a top elevational view of the apparatus of FIG. 21;

FIG. 23 is a side elevational view of the apparatus of FIG. 21;

FIG. 24 is a side elevational view of the apparatus of FIG. 21;

FIG. 25 is a cross section view of the apparatus of FIG. 21 taken along the line 25-25 shown in FIG. 22;

FIG. 26 is a perspective view of the apparatus of FIG. 21 with the guide member in an open state; and

FIG. 27 is a perspective view of the apparatus of FIG. 21 in a fully assembled state.

### DETAILED DESCRIPTION

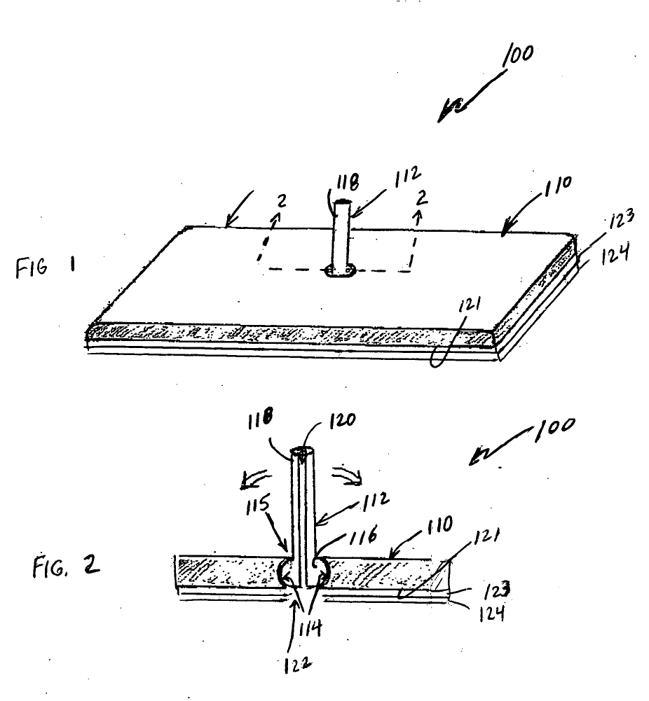

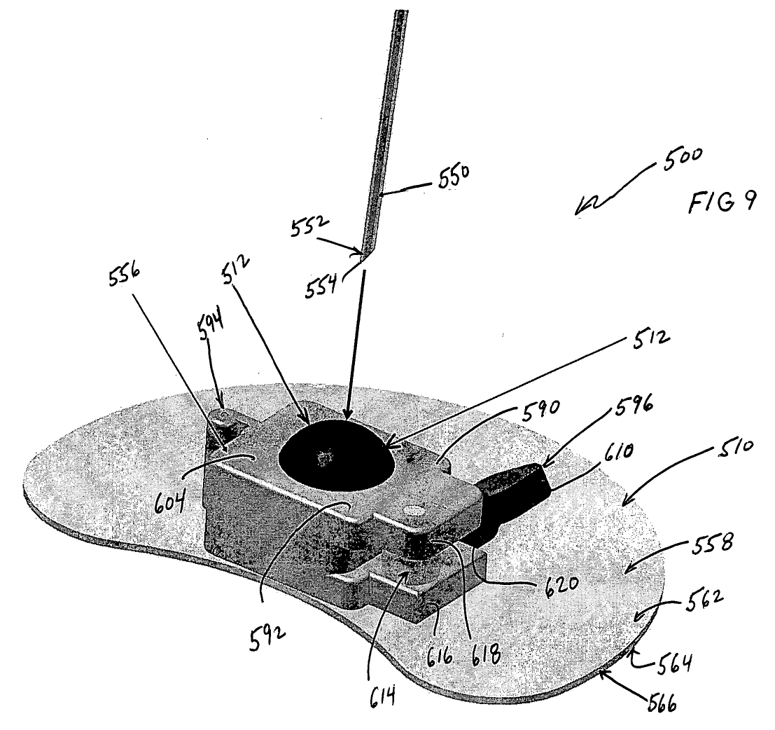

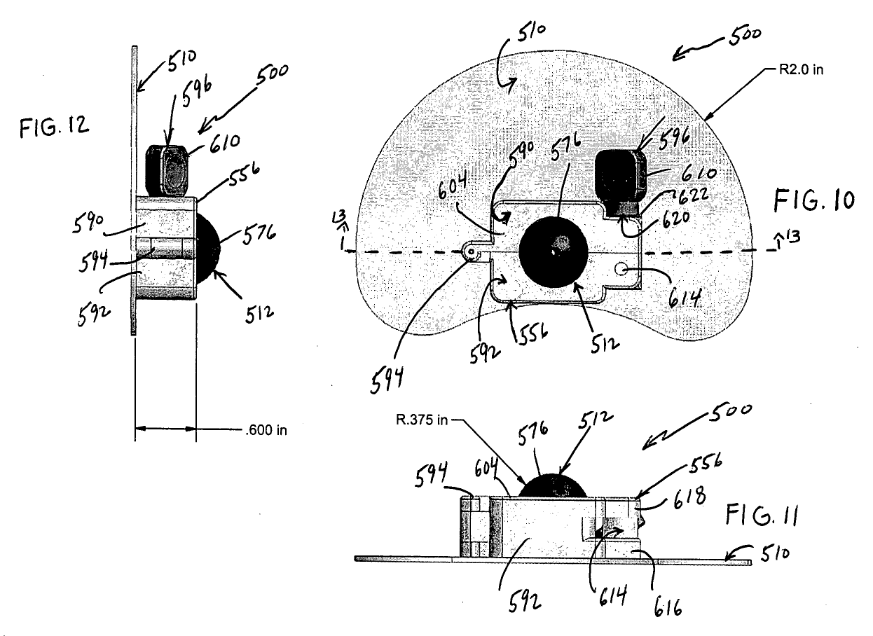

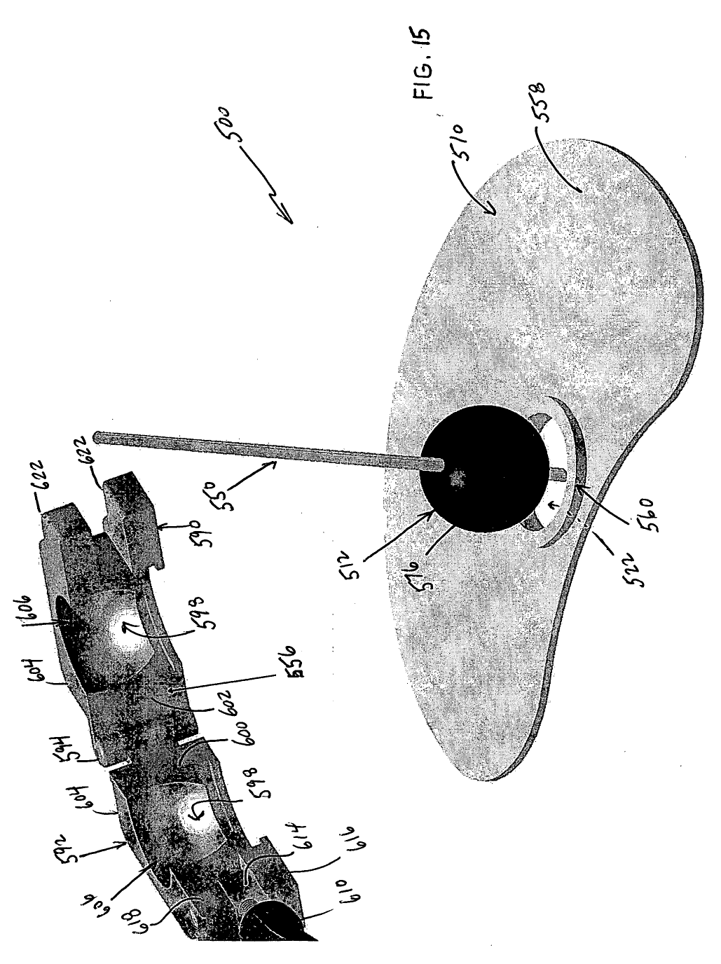

FIGS. 1-2 show a first embodiment of an apparatus 100 for supporting and/or guiding a device that is inserted, or desired to be inserted, into a body of a patient, for example, as part of a medical procedure. One example of such a device is a biopsy needle, an example of which is represented in FIG. 8 and described hereinbelow with respect to FIGS. 8-17. A biopsy needle may have, for example, a generally cylindrical profile and an insertion end that defines a tip, i.e., a portion of the needle that is to be inserted into the body of the patient.

The apparatus 100 includes a base 110 and a guide member 112. The base 110 is thin and flexible (or semi-flexible) and adapted to be positioned on the body of the patient. The base 110 has one or more surfaces 114 that define a seat 115 for the guide member 112. The guide member has two portions 116, 118. The first portion 116 has a shape that is, for example, generally spherical or ball-like. The second portion 118 is generally elongated and defines a longitudinally extending channel 120 that is adapted to provide at least some support for a device (e.g., a biopsy needle) to be inserted therein. The first portion 116 of the guide member 112 is pivotably mounted in the seat 115 defined by the base 110. This allows the orientation of the guide member 112 to be adjusted to place the device (e.g., a biopsy needle) in a desired orientation. The seat 115 has a size and shape that is substantially complementary to the portion of the guide member 112 that is seated therein.

The base 110 has a lower surface 121 that defines an opening 122 to allow passage of the device (e.g., a needle) therethrough. The lower surface 121 has adhesive 123 adhered thereto to allow the base 110 to be releasably attached to the skin of a patient. The adhesive 123 may be formed for example of a medical grade adhesive and/or other non-tissue reactive adhesive. The adhesive 123 may be chosen to provide that the base be firmly adherent to the skin yet easily removed therefrom when desired. The adhesive 123 may be formed in any way, for example, but not limited to, by applying a coating on one or more portions of the lower surface and/or by covering such portions with double sided tape (e.g., a flat substrate with two adhesive coated sides).

A removable release sheet 124 is disposed over the adhesive 123. The release sheet 124 is formed of a paper or plastic material that permits the release sheet to be manually peeled away from the adhesive 123 in order to expose the adhesive prior to attaching the base to the skin of the patient.

The base 110 may have any type of construction for example but not limited to, a homogeneous solid member, a non-homogeneous, semi-solid member with one or more pockets of gas and/or liquid, or a mesh (with a regular or an irregular topology) with openings that allow the flow of air therethrough.

In some embodiments, the base 110 may be malleable or bendable to be selectively manually bent, deformed or otherwise shaped and/or constructed to retain the form produced by such bending, deforming or shaping. This can be accomplished, for example, but not limited to, by embedding bendable metal strips or by forming the base of a deformable, shape retaining material.

The base 110 and the guide member 112 may each be formed in any manner, for example, but not limited to, by cutting (e.g., die cutting), injection molding, machining, casting, welding, and/or combinations thereto, and of any suitable material, for example, but not limited to, plastic, rubber, vinyl, polyethylene, polypropylene, carbon fiber, metal and/or combinations thereof. For example, in some embodiments, the base is formed of a die cut foam or bandage and the guide member is formed of injection molded polyethylene.

If desired, one or more portions of the apparatus 100 (or portions thereof) may be formed of a transparent, semi-transparent, or opaque material, or any combination thereof. Moreover, one or more of the parts of the apparatus (or portions of such parts) may include one or more radiopaque, or radiolucent, or other materials that are distinguishable in radiographic or other types of images in order to help medical personal or systems position the apparatus and/or device.

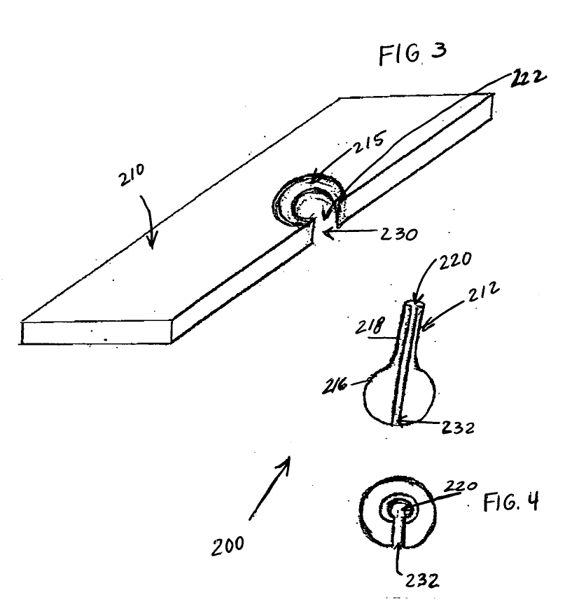

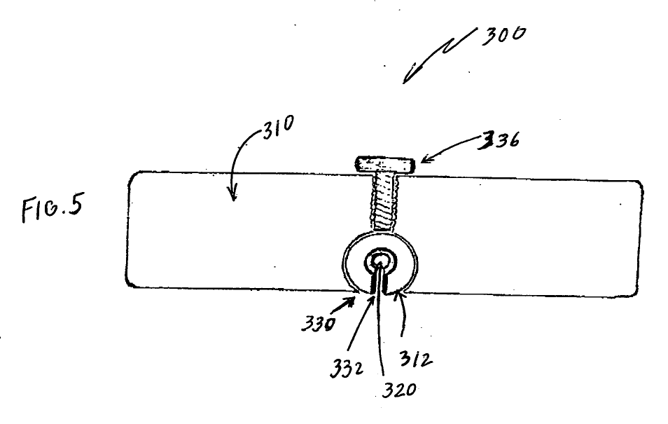

FIGS. 3-4 show another apparatus 200 for supporting and/or guiding a device that is inserted, or desired to be inserted, into the body of a patient. The apparatus 200 includes a base 210 and a guide member 212. The base 210 and the guide member 212 are similar to the base 110 and guide member 112 of the apparatus 100 described above with respect to FIGS. 1-2 with the exception that (1) a seat 215 for the guide member 212 is disposed at a marginal portion of the base 210, (2) the base 210 further defines a slot 230 that extends from the seat 215 to the marginal portion of the base 210 and (3) the guide member 212 includes a slot 232 that extends the length of the channel 220.

Positioning the guide member 212 at or near a marginal portion of the base 210 makes it possible for a probe or other instrument used in generating an image or other information to be placed closer to the device (e.g., needle) compared to that which would be possible if the guide member 212 were not disposed at a marginal portion of the base 210. This in turn assists in the use of image guided procedures and visualization of the angle of entry of the device (e.g., needle), which potentially leads to a faster, more accurate localization of the device (e.g., needle) into the intended target area, and thereby helps to reduce and/or eliminate the need for “trial and error” methods of positioning the needed and speeds up the medical procedure.

Providing the slot 230 in the base 210 and the slot 232 in the guide member 212 enables the apparatus 200 to be separated from a device (e.g., a needle) inserted into a patient, without the need to remove the device (e.g., a needle) from the body of the patient. In this embodiment for example, the device (e.g., a needle) is separated from the apparatus by way of the slot 232 in the guide member and the slot 230 in the base.

The apparatus 200 (or one or more portions thereof) may be pre-assembled and shipped in a sterile package or container (not shown), in order to lessen the chance of infection for patients, physicians and/or technologists. The apparatus 200 may be a single-use disposable device in order to further lessen the chance of infection.

One example of the manner in which the apparatus 200 may be used is as follows. After localizing the site of a biopsy, or the entry point for a drainage procedure, a physician may mark and clean an area on a patient's skin. The apparatus 200 is then placed over the marked site, with the seat 215 for the guide member 212 and the opening 222 in the base positioned over the desired entry point on the patient's skin. With firm application, the device (e.g., needle) is inserted into the guide member 212 and slowly advanced, for example, in response to information provided by computerized tomography (CT) and/or ultrasound images. Positioning the guide member 212 at or near the marginal portion (e.g., the edge) of the base 210 allows an ultrasound transducer (not shown) to easily detect the device (e.g., a needle) throughout the procedure. As the device (e.g., a needle) is advanced, the orientation of the guide member 212 can be adjusted so as to obtain the desired orientation of the device (e.g., a needle) and thereby cause the device (e.g., a needle) to arrive at the desired position within the patient's body. A desired biopsy sample may thereafter be obtained. If drainage is to be performed, the needle is placed in position and a guidewire (not shown) is advance through the needle and into the lesion that is to be drained. The apparatus 200 may then be removed from around the needle and the wire, allowing for placement of dilators and/or a catheter drain (not shown).

FIG. 5 shows another apparatus 300 for supporting and/or guiding a device inserted, or desired to be inserted, into the body of a patient. The apparatus 300 includes a base 310 and a guide member 312. The base 310 and the guide member 312 are similar to the base 210 and guide member 212 of the apparatus 200 described above with respect to FIG. 3-4 with the exception that the apparatus 300 further includes a clamp 336, a fastener (e.g., a thumbscrew) and/or other mechanism for releasably retaining the guide member 312 in the desired orientation.

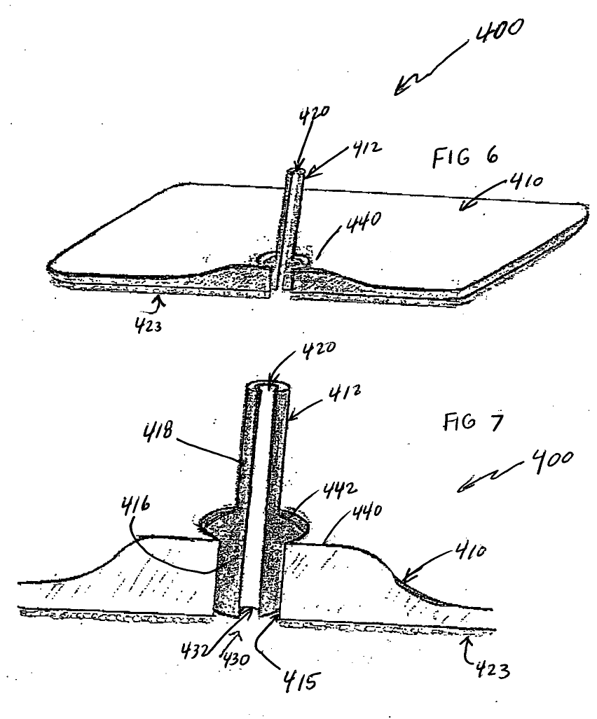

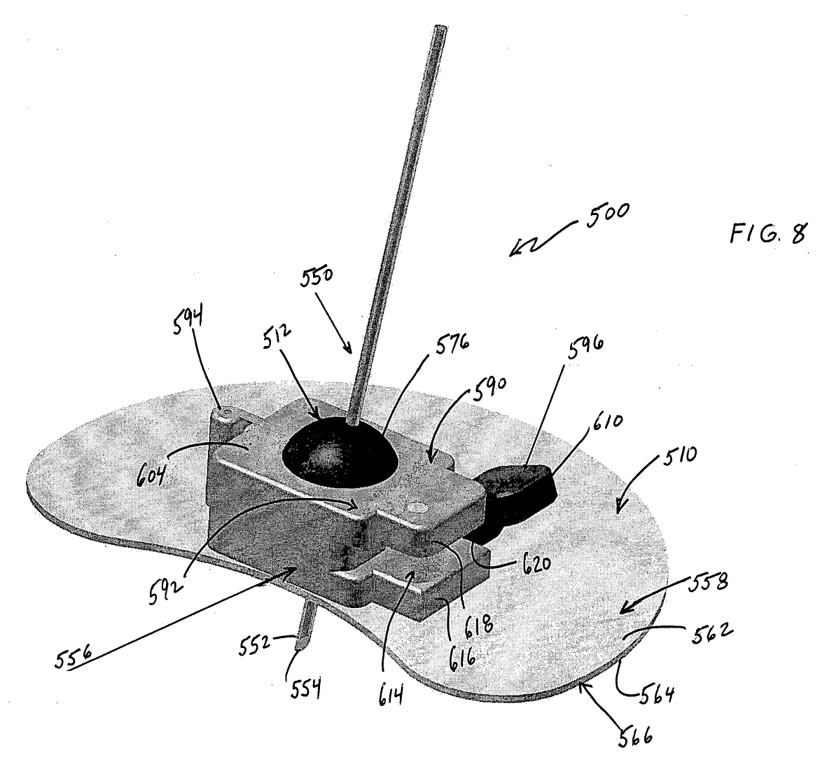

FIGS. 6-7 show another apparatus 400 for supporting and/or guiding a device inserted, or desired to be inserted, into the body of a patient. The apparatus 400 includes a base 410 and a guide member 412. The base 410 and the guide member 412 are similar to the base 210 and guide member 212 of the apparatus 200 described above with respect to FIGS. 3-4 with the exception that (1) the lower portion 416 of the guide member 412 and the seat 415 defined by the base 410 are each generally cylindrical and (2) the base 410 defines a raised portion 440 that is disposed semi-circumferentially about the seat 415 and (3) the guide member further defines a shoulder 442 disposed between the first portion 416 and the second portion 418 and seated on the raised portion of the base 410.

FIGS. 8-17 show another embodiment of an apparatus 500 for supporting a device inserted, or desired to be inserted, into a body of a patient, for example, as part of a medical procedure. One example of such a device is a biopsy needle 550, an example of which is represented in FIG. 8. The biopsy needle 550 may have, for example, a generally cylindrical profile and an insertion end 552 that defines a tip 554, i.e., a portion of the needle that is to be inserted into the patient.

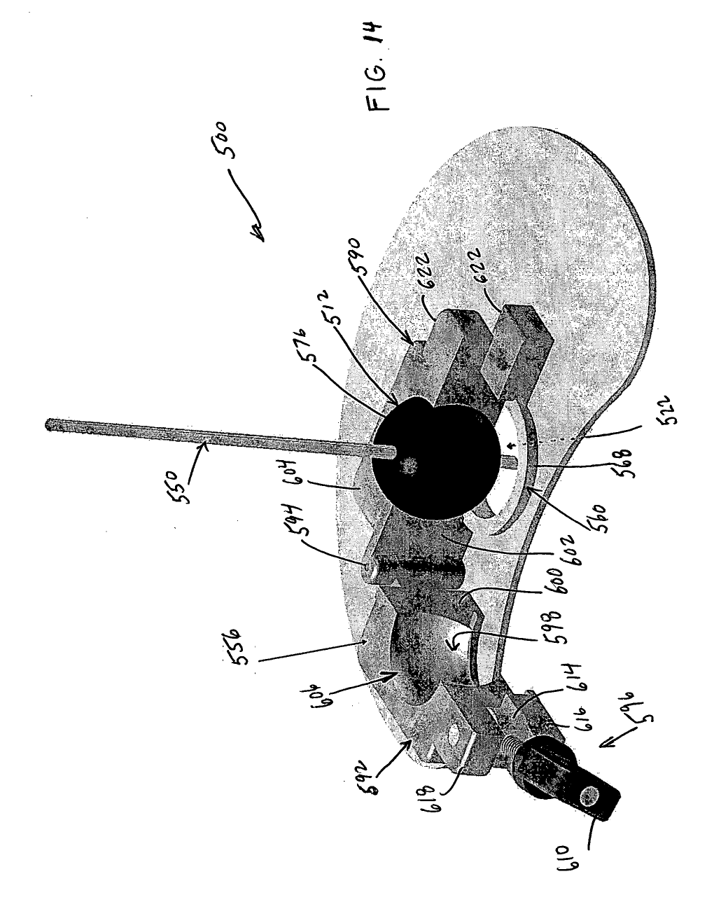

The apparatus 500 includes a base 510, a guide member 512 and a guide support 556. The base 510 has two portions 558, 560 (FIGS. 14-17). The first portion 558 is a generally flexible or semi-flexible platform (e.g., a mat or pad) and is adapted to be positioned on the body of the patient and generally help stabilize the other components of the apparatus 500. The first portion 558 may be kidney shaped, as shown, or any other shape that is usable for the medical procedure to be performed on the patient, for example, but not limited to, semi-circular, circular, rectangular, and other regular and irregular shapes.

The first portion 558 of the base has upper and lower surfaces 562, 564 and one or more peripheral surfaces 566. The upper surface 562 supports the guide support 556. One or more portions of the lower surface 564 have adhesive (e.g., similar to adhesive 123 (FIG. 1)) adhered thereto so as to allow the base 510 to be releasably attached to the skin of the patient. The adhesive may be formed for example of a medical grade adhesive and/or other non-tissue reactive adhesive. The adhesive may be chosen to provide that the base be firmly adherent to the skin yet easily removed therefrom if desired. The adhesive may be applied in any way, for example, but not limited to, by applying a coating on one or more portions of the lower surface and/or by covering such portions with double sided tape (e.g., a flat substrate with two adhesive coated sides). A removable release sheet (e.g., similar to release sheet 124 (FIG. 1)) may be disposed over the adhesive. The release sheet is formed of a paper or plastic material that permits the release sheet to be manually peeled away from the adhesive in order to expose the adhesive prior to attaching the base to the skin of the patient.

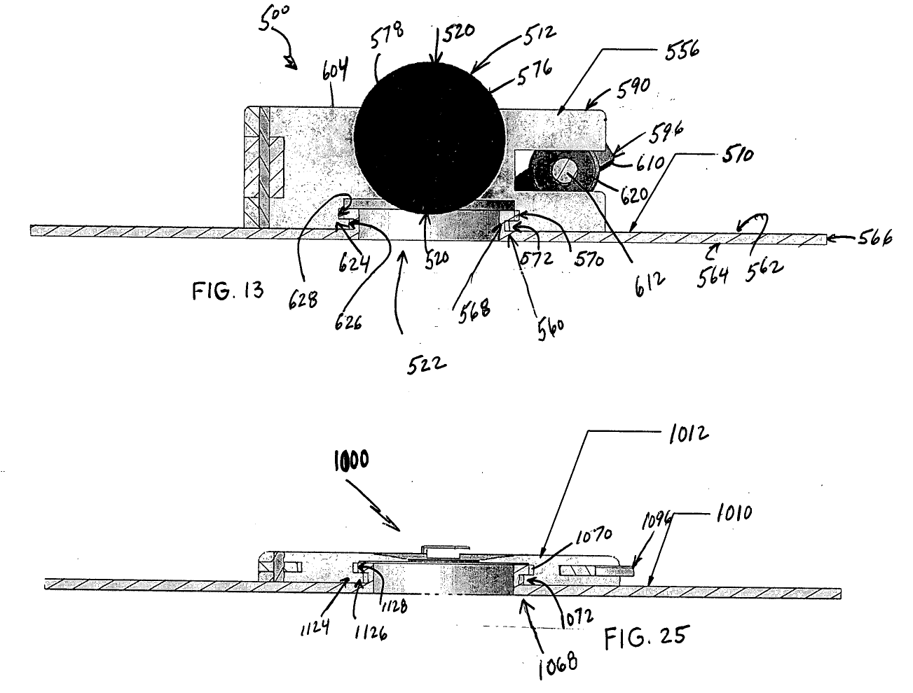

The second portion 560 of the base defines a mount 568 that is adapted to be engaged by the guide support 556 to releasably retain the guide support 556 thereto. The mount 568 projects, for example, upward from the upper surface 562 of the base 510 and includes a flange 570 (FIG. 13) that extends substantially parallel to the upper surface 562. A recess 572 (FIG. 13) is defined between the flange 570 (FIG. 13) and the upper surface 562 of the base 510. The mount 568 may be annular as shown although this is not required.

The first portion 558 of the base 510 further defines an opening 522 (FIGS. 13-17) to allow the tip 554 of the device 550 to pass therethrough. The opening 522 is preferably circular and large enough to permit the tip of the device to pass therethrough, regardless of the angle of orientation of the device (e.g., over the full range of possible angular orientations of the device within the apparatus), although this is not required.

The two portions 558, 560 (FIGS. 13-17) of the base 510 may be formed in any manner, for example, but not limited to, by cutting (e.g., die cutting), injection molding, machining, casting, welding, and/or combinations thereto, and of any suitable material, for example, but not limited to, plastic, rubber, vinyl, polyethylene, polypropylene, carbon fiber, metal and/or combinations thereof. Moreover, the two portions 558, 560 may be formed separately and thereafter fixedly attached to one another, for example, but not limited to, by bonding, gluing, heat sealing, pressing, fastening, or welding (e.g., ultrasonic welding). For example, in some embodiments, the first portion is formed of a die cut foam or bandage and the mount is formed of injection molded polyethylene that is thereafter glued or hot sealed to first portion. The portions 558, 560 may alternatively be formed in an integral fashion, for example, as a single piece, or further alternatively, in a build-up fashion, for example, by over molding, where one portion is formed and concurrently joined to a portion previously formed.

The base 510 may have any type of construction for example but not limited to, a homogeneous solid member, a non-homogeneous, semi-solid member with one or more pockets of gas and/or liquid, or a mesh (with a regular or an irregular topology) with openings that allow the flow of air therethrough. In some embodiments, the base 510 may be malleable or bendable to be selectively manually bent, deformed or otherwise shaped and/or constructed to retain the form produced by such bending, deforming or shaping. This can be accomplished, for example, but not limited to, by embedding bendable metal strips or by forming the base 510 of a deformable, shape retaining material.

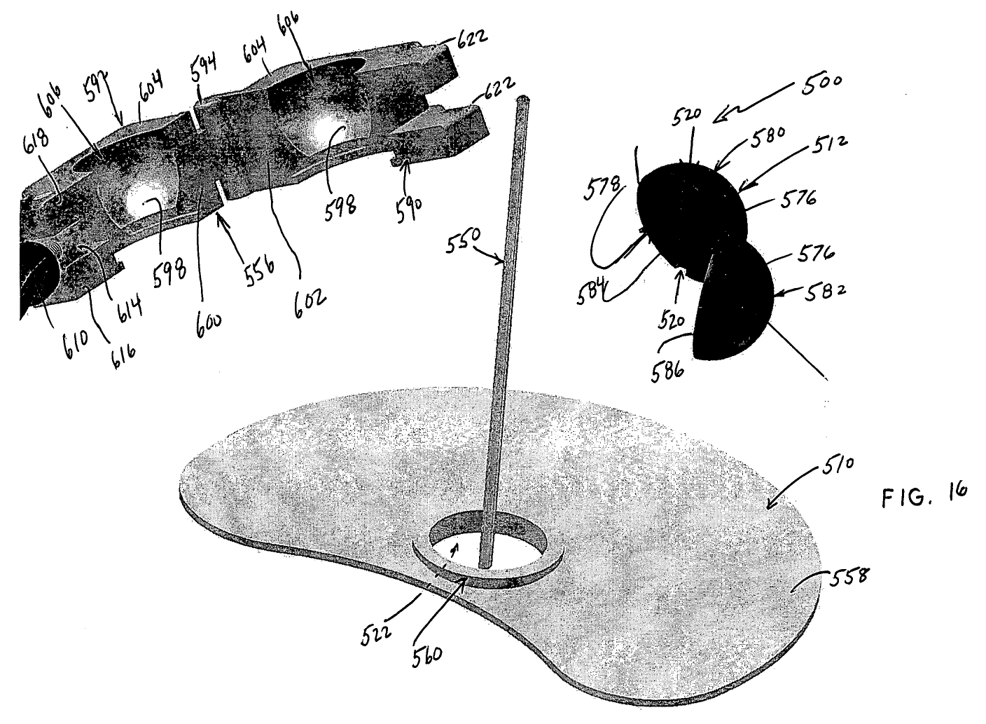

The guide member 512 is adapted to provide at least some support and/or guidance for the device 550 inserted, or to be inserted into the body of the patient. The guide member 512 includes one or more exterior surfaces 576 that define a generally spherical shape, which facilitates rotation of the guide member 512. The guide member 512 further includes one or more interior surfaces 578 (FIGS. 13, 16) that define a channel 520 extending therethrough. The channel 520 is adapted to receive one or more portions of the device 550 to be inserted into the body of the patient. To this effect, the channel 520 may have a size and shape that is substantially complementary to one or more portions of the device 550. For example, to accommodate the biopsy needle 550, the channel 520 may have a substantially cylindrical shape. The width of the channel 520 may be selected, for example, to be large enough to receive the desired portion(s) of the device yet small enough that one or more of the interior surfaces 578 (FIGS. 13, 16) provide at least some lateral support for the device 550.

FIG. 16 is a perspective view of the apparatus with the guide member 512 and guide support 556 shown in an open state. Referring now to FIG. 16, the guide member 512 is formed of two separate portions 580, 582 (FIG. 16) (e.g., hemispheres) that are pivotably connected to one another. The first portion 580 (FIG. 16) has a surface 584 (FIG. 16) that defines one side of the channel 520. The second portion 582 (FIG. 16) has a surface 586 (FIG. 16) that defines the other side of the channel 520. The surfaces 584, 586 are approximately parallel to one another when the guide member 512 is in a closed position. Such surfaces may be referred to herein as mating surfaces even though the surfaces may or may not contact one another when the guide member is in a closed position.

The guide support 556 is adapted to releasably capture the guide member 512 and to releasably retain the guide member 512 to the base 510. The guide support 556 has two portions 590, 592 that are to some extent mirror images of one another. The two portions 590, 592 may be pivotably connected to one another, for example, by a hinge 594 disposed at one end of the two portion 590, 592. The two portions 590, 592 may alternatively be formed with an integral living hinge connecting the two portions 590, 592. A clamp 596 is disposed, for example, at a free end of the two portions 590, 592, may be used to releasably fasten the free end of the two portions 590, 592 in order to reduce relative movement therebetween.

The two portions 590, 592 of the guide support 556 include surfaces that collectively define a seat 598 (FIGS. 14-16) for the guide member 512. Portions 600, 602 (FIGS. 14-16) of these surfaces are approximately parallel to one another when the guide member 512 is in a closed position. The seat 598 (FIGS. 14-16) may have a size and shape that is substantially complementary to the portion of the guide member 512 seated therein. To releasably capture the guide member 512, the guide support 556 is first placed in the open state and the guide member 512 is positioned in one or more portions of the seat 598 (FIGS. 14-16). The guide support 556 is then placed in a closed state wherein the surfaces 600, 602 (FIGS. 14-16) are positioned approximately parallel to one another. The clamp 596 may then be engaged to retain the guide support 556 in the closed state.

An upper surface 604 of the two portions 590, 592 defines an opening 606 for receiving the guide member 512. The opening 606 has a size and/or shape that is, for example, large enough to permit the full range of desired angular orientations of the device 550, yet small enough to prevent the guide member 512 from becoming separated from the guide support 556 while the guide support is in a closed state.

The clamp 596 includes a thumb screw 610 with a threaded end 612 (FIG. 13) adapted to releasably engage a threaded opening defined by an insert 614. The insert 614 is pivotably mounted between two spaced apart flanges 616, 618 defined by the second portion 592. The thumb screw 610 further includes bearing surfaces 620 adapted to contact one or more bearing surfaces 622 disposed on the first portion 590. The operation of the clamp 596 is as follows. With the guide support 556 positioned in a closed state, the thumb screw 610 is threaded into the opening defined by the insert 614 and positioned so that the bearing surfaces 620 are in register with the bearing surfaces 622 of the first portion 590. The thumb screw 610 may thereafter be tightened to cause the bearing surfaces 620 of the thumb screw 610 to contact the bearing surfaces 622 of the first portion 590, thereby forcing the two portions 590, 592 of the guide support 556 toward one another. Further tightening the thumb screw 610 causes the magnitude of the clamping force to increase. Loosening the thumb screw 610 decreases the magnitude of the clamping force. After the bearing surface 620 of the thumb screw 610 no longer contacts the bearing surface 622 of the first portion 590, the two portions 590, 592 of the guide support 556 may be pivoted apart to some extent, to thereby place the guide support 556 in a partially open state. If the thumb screw 610 is completely removed from the insert 614, or pivoted in a clockwise direction to so that the bearing surface 620 of the thumb screw 610 is no longer in register with the bearing surfaces 622 of the first portion 590, then the two portions 590, 592 of the guide support 556 may be further pivoted apart from one another to place the guide support 556 in a fully open state.

The clamp and the seat are shaped and sized so as to provide a first clamp position at which the clamp provides a force that is small enough to allow the guide member to be rotated to a desired position in response to an ordinary amount of force applied by medical personnel, and a second clamp position at which the clamp provides enough force to cause the seat to contact the guide member with enough force to prevent the guide member from moving in response to the ordinary amount of force applied by medical personal, such that the guide member is locked in position.

The guide support 556 further defines a catch 624 (FIG. 13) that engages the mount of the base 610 to releasably retain the guide support 556 thereto. The catch 624 (FIG. 13) may include, for example, a flange 626 (FIG. 13) and a recess 628 (FIG. 13). The flange 626 (FIG. 13) is received by the recess 572 (FIG. 13) between the upper surface of the base and the flange 570 (FIG. 13) of the mount 568 (FIG. 13). The recess 628 (FIG. 13) receives the flange portion 570 (FIG. 13) of the mount 568 (FIG. 13). The recess may be annular as shown although this is not required.

In this embodiment, the guide support 554 is disposed at or near a marginal portion (e.g., an edge) of the base 510. This enables a probe or transducer or source used in generating an image or other information to be placed closer to the device 550 than would otherwise be possible if the guide support 554 was located in the center of the base 510. Being able to place a probe or other source closer to the device 550 aids in the use of image guided procedures and visualization of the orientation and tract of the device. This in turn may lead to a faster, more accurate localization (i.e., the directing of the device (e.g., needle) into the intended target area) thereby reducing and/or eliminating the need for “trial and error” methods and speeding up the medical procedure.

One advantage of the embodiment described in FIGS. 8-17 is that the guide member can be separated from a device that is inserted into a patient without the need to remove the device from the patient. Another advantage is that the guide member and the guide support may each be separated from a device that is inserted into a patient without the need to remove the device from the patient. Another advantage is that the guide support can be disengaged from the base by adjusting the position of the pivotably connected portions of the guide support. Another advantage is that the guide member is releasably captured in the guide support by adjusting the position of the pivotably connected portions thereof.

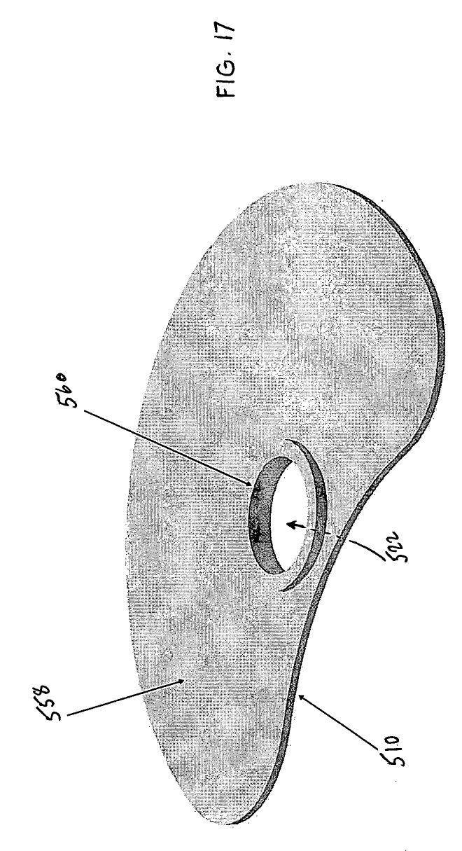

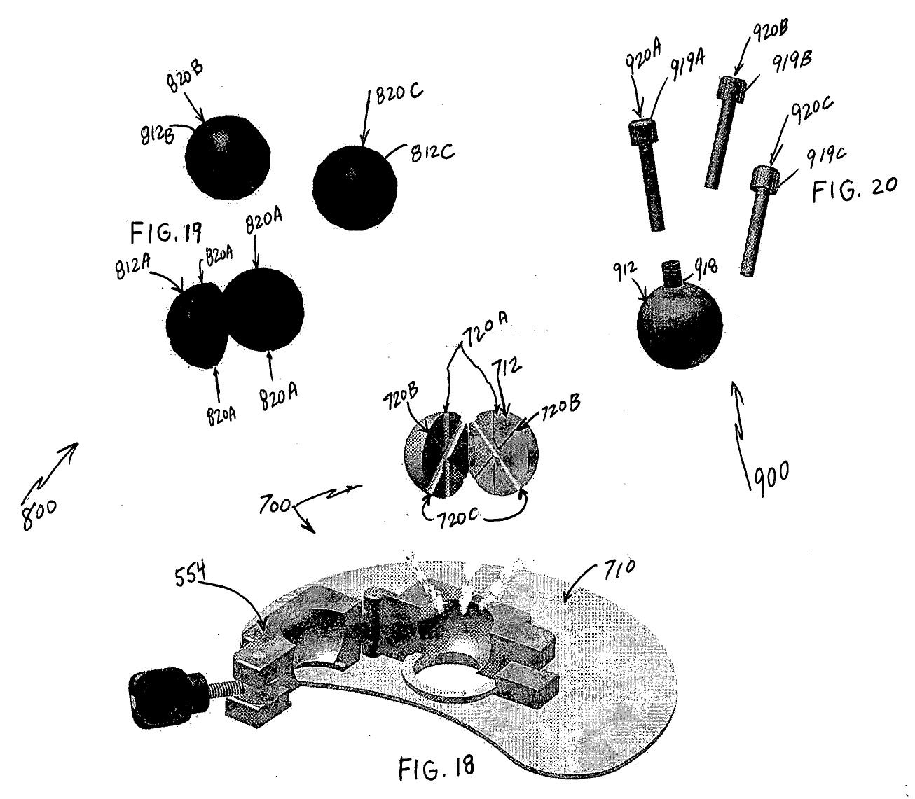

FIG. 18 shows another apparatus 700 for supporting and/or guiding a device that is inserted, or to be inserted, into the body of a patient. The apparatus includes a base 710 and a guide member 712. The base is substantially the same as the base 510 described hereinabove with respect to the apparatus of FIGS. 8-17. The guide member 712 is similar to the guide member 512 described hereinabove with respect to FIGS. 8-17, with the exception that the guide member 712 defines a plurality of different sized channels 720A-720C, in contrast to the guide member of FIGS. 8-17 which defines only one channel. Each of the channels 720A-720C may extend, for example, approximately through the geometric center of the guide member 512, although this is not required. The availability of different sized channels makes it easier to accommodate devices (e.g., needles) of different size. For example, the operator may select the channel that is best suited for the particular device (e.g., needle) that is to be used. This in turn, may help improve the accuracy and reproducibility of the positioning of the needle and the intended biopsy tract.

FIG. 19 shows another embodiment 800 for providing different sized channels. This embodiment includes a plurality of guide members 812A-812C. Each of the plurality of guide members 812A-812C is similar to the guide member described hereinabove with respect to FIGS. 8-17, with the exception that the plurality of guide members 812A-812C each defines a channel 820A-820C, respectively, that is different in size than the channels defined by the other guide members of the plurality of guide members 812A-812C. As stated above, the availability of different sized channels makes it easier to accommodate devices (e.g., needles) of different size. For example, the operator may select the guide member with the channel that is best suited for the particular device (e.g., needle) that is to be used. This in turn, may help improve the accuracy and reproducibility of the positioning of the needle and the intended biopsy tract.

FIG. 20 shows another embodiment 900 for providing different sized channels. This embodiment 900 includes a single guide member 912 and a plurality of inserts 919A-919C. Instead of a channel, the guide member 912 defines a seat 918 adapted to receive any one of the plurality of inserts 919A-919C. Each of the inserts 919A-919C defines a channel 920A-920C, respectively, that similar to the channel 520 defined by the guide member 512 described hereinabove with respect to FIGS. 8-16, with the exception that each channel 920A-920C has a size that is different than the sizes of the other channels defined by the other inserts of the plurality of inserts 919A-919C. The guide member further includes a feature (e.g., a threaded sleeve) that is adapted to releasably engage a corresponding feature (e.g., a threaded cap) of the insert seated on the seat. As stated above, the availability of different sized channels makes it easier to accommodate devices (e.g., needles) of different size. For example, the operator may select the insert that is best suited for the particular device (e.g., needle) that is to be used. This in turn, may help improve the accuracy and reproducibility of the positioning of the needle and the intended biopsy tract.

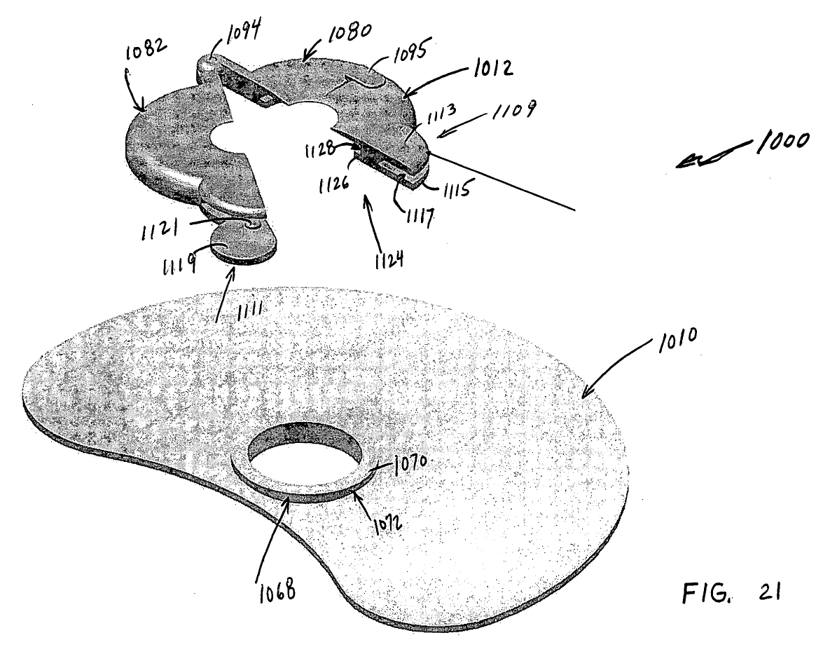

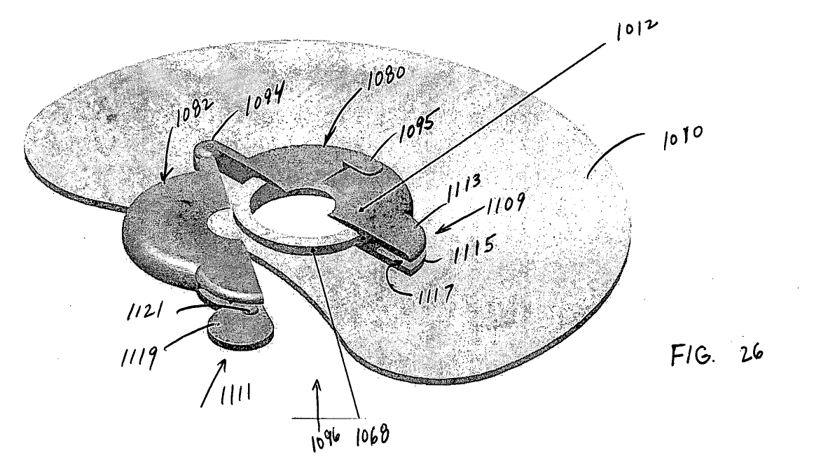

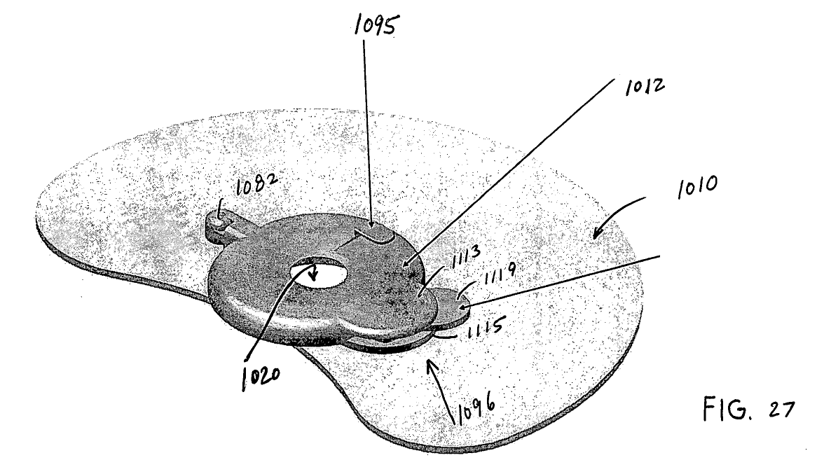

FIGS. 21-27 show another apparatus 1000 that may be used to support a device (e.g., see needle 550 (FIG. 8)) inserted, or desired to be inserted, into a body of a patient. The apparatus 1000 includes a base 1010 and a guide member 1012. The base 1010 is substantially the same as the base 510 described above with respect to the apparatus 500 shown in FIGS. 8-17. The guide member 1012 has some similarity to the guide member 512 described hereinabove in that the guide member 1012 is also adapted to provide at least some support and/or guidance for a device (e.g., see needle 550 (FIG. 8)) inserted, or desired to be inserted into a patient. However, unlike the guide member 512 described hereinabove with respect to FIGS. 8-17, the guide member 1012 does not require a guide support similar to guide support 556. Rather, the guide member 1012 is adapted to be attached directly to the base 1010. More specifically, the guide member 1012 defines a catch 1124 that engages the mount 1068 of the base 1010 to releasably retain the guide member 1012 to the base 1010. The catch 1124 is similar to the catch 624 (FIG. 13) defined by the guide support 556. The catch 1124 includes, for example, a flange 1126 and a recess 1128. The flange 1126 is received by the recess 1072 defined between the upper surface of the base 1010 and the flange of the mount 1068. The recess 1128 receives the flange portion 1070 of the mount 1068. The flange 1126 and recess 1128 may be annular as shown although this is not required.

Because the base in the apparatus is substantially the same as the base described above with respect to the apparatus shown in FIGS. 8-17, it is possible to change from the support mechanism shown in FIGS. 8-17 to the support mechanism shown in FIGS. 21-27 by simply removing the guide member and guide support from the apparatus of FIGS. 8-17 and installing the guide member shown in FIGS. 21-27. Thus there is provided the ability to replace a support without the need to remove the base.

Moreover, the guide member 1012 has a profile that is a significantly lower profile than the combined profiles of the guide member 512 and the guide support 556. The guide member 1012 may be used, for example, to support a different type of device (e.g., a catheter) or procedure than that supported by the guide member 512 described with respect to FIGS. 8-17.

Thus, it is possible to change the type of support provided without the need to remove the base from the patient.

The guide member 1012 is further described below.

The guide member 1012, as with the guide member 512 (FIGS. 8-17), defines a channel 1020, which is adapted to receive one or more portions of the device inserted, or desired to be inserted, into the patient. The size and/or shape of the channel may be, for example, large enough to receive one or more portion(s) of the device yet small enough that one or more of the surfaces of the guide member provides at least some support and/or guidance for the device. Although the channel 1020 of the guide member 1012 appears wider than the channel 520 of the guide member 512 described hereinabove, this is not a requirement.

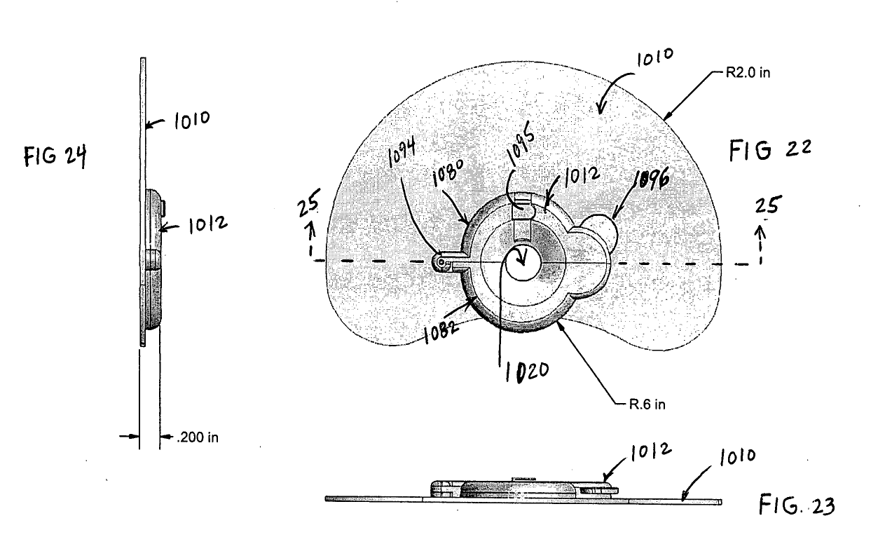

Referring to FIG. 22, it can be seen that the guide member 1012 includes two portions 1080, 1082, which are, to some extent, mirror images of one another. One portion 1080 of the guide member 1012 defines one side of the channel 1020. The second portion 1082 of the guide member 1012 defines the other side of the channel 1020. The two portions 1080, 1082 of the guide memberl012 are pivotably connected to one another, for example, by a hinge 1094 disposed at one end of the two portions 1080, 1082. A snap lock is disposed, for example, at a free end of the two portions 1080, 1082, and may be used to releasably fasten the free end of the two portions in order to reduce relative movement therebetween.

The guide member further includes a guide clip 1095 and a fastener 1096. The guide clip 1095 is used to guide a device (e.g., a catheter. The fastener 1096 is used to releasably fasten the two portions of the guide member 1080, 1082 and thereby retain the guide member in a closed state. The fastener 1096 is made up of a catch 1109 fixedly connected to the first portion 1080 of the guide member and a latch 1111 connected to the second portion 1082 of the guide member. The catch 1109 includes two flanges 1113, 1115 that are spaced apart from one another and define a recess 1117 therebetween. The latch 1111 includes a flange 1119 that is pivotably connected to the second portion 1082 of the guide member 1012. A boss 1121 projects from the surface of flange 1119. The operation of the fastener 1096 is as follows. With the guide member 1012 positioned in a closed state, the latch 1111 is pivoted such that the flange 1119 is received in the recess 1117 defined between the two flanges 1113, 1115 and the boss 1121 engages a recess (not shown) defined by the lower surface of the flange 1113 to retain the guide member 1012 in a closed state. The fastener 1096 is released by pivoting the latch 1111 in a direction out of the recess 1117, such that the boss 1121 disengages from the recess (not shown) in the flange 1113. The two portions of the guide member 1012t may then be pivoted apart from one another to place the guide member 1012 in an open state.

The guide member 1012 is attached to the base 1010 as follows. The guide member 1012 is first positioned on the base 1010 and adjusted so as to be in the open state. One portion of the guide member 1012 is positioned such that the associated portion of the catch 1124 engages a respective portion of the mount 1068. The other portion of the guide member 1012 is then pivoted parallel to the base 1010 thereby placing the guide member in a closed position and causing the second portion of the catch 1124 to engage the respective portion of the mount 1068. The fastener 1096 is then engaged to retain the guide member 1012 in such state.

One advantage of the embodiment described in FIGS. 8-17 is that the guide member can be separated from a device that is inserted into a patient without the need to remove the device from the patient. Another advantage is that the guide member and the guide support may each be separated from a device that is inserted into a patient without the need to remove the device from the patient. Another advantage is that the guide support can be disengaged from the base by adjusting the position of the pivotably connected portions of the guide support. Another advantage is that the guide member is releasably captured in the guide support by adjusting the position of the pivotably connected portions thereof.

One advantage of the embodiment described in FIGS. 22-27 is that the guide member can be separated from a device that is inserted into a patient without the need to remove the device from the patient. Another advantage is that a single base can accommodate different types of guide members, thereby making it possible to remove the support used in the course of a procedure in order substitute another type of support, without the need to remove the base that connects the support to the body of the patient. Another advantage is that the guide member and the guide support may each be separated from a device that is inserted into a patient without the need to remove the device from the patient. Another advantage is that the guide support can be disengaged from the base by adjusting the position of the pivotably connected portions of the guide support. Another advantage is that the guide member is releasably captured in the guide support by adjusting the position of the pivotably connected portions thereof.

It should be understood, that although the guide member is shown having a pancake-like profile, the guide member is not limited to such. Indeed, in some embodiments, the profile shown in FIGS. 21-27 may be too low to provide the amount of support desired for a catheter.

Note that the any of the apparatus (or one or more portions thereof) may be preassembled and shipped in a sterile package. Moreover, the apparatus may be a single-use disposable device. These measures may help lessens the chance of infection for patients, physicians and technologists.

Also note that one or more portions of the apparatus (or portions thereof) may be formed of a transparent, semi-transparent, or opaque material, or any combination thereof. Moreover, one or more of the parts of the apparatus (or portions of such parts) may include one or more radiopaque, or radiolucent, or other materials that are distinguishable in radiographic or other types of images in order to help medical personal or systems position the apparatus and/or device.

The methods and/or apparatus described herein may be employed in, for example, but not limited to, guided biopsies (e.g., CT (“computerized tomography”) guided biopsies, MR (“magnetic resonance”) guided biopsies, ultrasound guided biopsies, and/or combinations thereof), guided drainage of fluid (e.g., CT, MR Ultrasound and/or combinations thereof), image guided placement of a device (e.g., a needle, catheter or wire) into a body, image guided placement of marker (e.g., a radiopaque or radiolucent tissue marker) and image guided placement of implants (e.g., radioactive seeds, particles for treatment of malignant disease). Various embodiments examples demonstrating the use one or more of the methods and apparatus disclosed herein are described hereinafter.

An example of the manner in which the apparatus may be used in association with CT equipment is as follows. Using CT scanning equipment, the radiologist or surgeon is able to pinpoint a specialized biopsy needle into the substance of a suspicious lesion or fluid collection seen on previous diagnostic radiology studies. After the area of the lesion is localize, the overlying skin is then prepped and draped in sterile fashion. The base is placed over the intended skin entry point, firmly adhering to the skin. The skin is then anesthesized, for example, with 1% Lidocaine. A small nick is made in the skin and the guide member and guide support are assembled onto the base. A needle is gently set into the proper size hole in the guide member at the expected angle of entry. A CT image is obtained and the needle is visualized at it's angle of trajectory towards the intended target. If the angle is appropriate, the thumb screw (Clamp) is tightened. The needle is then advanced into the patient, to the appropriate depth of the intended lesion.

For biopsies, the needle is generally a co-axial type, through which a smaller biopsy needle is passed. Multiple biopsy samples are taken, and the procedure is complete. The apparatus is removed entirely, and pressure is held over the wound for 5-10 minutes.

For drainage of a fluid collection or abscess, the needle is advanced into the collection. Through this needle, a wire is passed, coiling within the cavity of the collection to gain “purchase” within the body. The needle is then withdrawn, taking care not to remove the wire. The guide member and guide support are removed, leaving the wire in place. Over the wire, a series of dilators are passed to the depth of the fluid collection, creating a tract. Finally, the selected drainage catheter is passed over the wire, coiling within the collection. The wire is removed and the catheter is aspirated, to ensure that fluid is withdrawn and that the catheter is functioning properly. The guide member 1012 (sometimes referred to herein as a catheter retention module) is then fitted onto the and positioned around the exiting drainage catheter. The guide clip is then locked over the catheter, holding it firmly in place. The catheter is then connected to a drainage bag, and the procedure is complete.

An example of the manner in which the apparatus may be used in association with ultrasound imaging is as follows. Using ultrasound imaging equipment the radiologist or surgeon is able to pinpoint a specialized biopsy needle into the substance of a suspicious lesion or fluid collection seen on previous diagnostic radiology studies. After scanning the patient with ultrasound, the area of the lesion is localized and the overlying skin is then prepped and draped in sterile fashion. The base of the apparatus is placed over the intended skin entry point, firmly adhering to the skin. The skin is then anesthesized with 1% Lidocaine. A small nick is made in the skin and the guide member and guide support are fitted into place on the base. A needle is gently set into the proper size hole in the guide member at the expected angle of entry, and slowly advanced into the patient. The ultrasound transducer is placed up against the base and edge of the guide member and guide support, allowing for easier visualization of the needle. Using real-time ultrasound imaging, the physician is able to “see” the needle advance into the targeted area. Since the apparatus is holding the needle in place, the physician is able to use one hand to hold the ultrasound transducer and the other hand to gently advance the needle. Once the proper trajectory angle is obtained, the clamp is tightened and the needle is followed into the intended lesion.

For biopsies, the needle is generally a co-axial type, through which a smaller biopsy needle is passed. By locking the needle in place, the apparatus provides extra “hands” for the radiologist, who can let go of the needle and set up for the rest of the procedure. The needle won't move or deflect, so there is less chance of misdirection. One of the radiologists' hands holds the transducer, while the other hand is free to manipulate the needle and fire the biopsy gun. The extra “pair of hands” comes in very handy with deep or hard-to-reach lesions in the body. Multiple biopsy samples are taken, and the procedure is complete. The apparatus is removed entirely, and pressure is held over the wound for 5-10 minutes.

For drainage of a fluid collection or abscess, the needle is advanced into the collection. Through this needle, a wire is passed, coiling within the cavity of the collection to gain “purchase” within the body. This process is facilitated by the apparatus, as the physician no longer needs to a.) hold the transducer, b.) position the needle and c.) advance the wire into the fluid collection. Such a process generally requires three hands, meaning that an assistant often is required to assist. By locking the needle in place, the apparatus provides extra “hands” for the radiologist, who can let go of the needle and set up for the rest of the procedure. After the wire is in the fluid collection, the outer needle is then withdrawn, taking care not to remove the wire. The guide member and guide support are removed, leaving the wire in place. Over the wire, a series of dilators are passed to the depth of the fluid collection, creating a tract. Finally, the selected drainage catheter is passed over the wire, coiling within the collection. The wire is removed and the catheter is aspirated, to ensure that fluid is withdrawn and that the catheter is functioning properly. The guide member is then fitted into the base, positioned around the exiting drainage catheter. The guide clip is then locked over the catheter, holding it firmly in place. The catheter is then connected to a drainage bag, and the procedure is complete.

It should be understood that the various aspects of the present invention are not limited to the embodiments described above, except where otherwise stated. For example, although the guide member and guide support are each shown having two separate portions that are pivotally connected to one another, the guide member and guide support may each be formed of any number of portions. If there is more than one portion, the portions may be connected (in any manner) and/or completely separable from one another. In addition, although the guide member is shown as generally spherical with a generally cylindrical channel, the guide member may take other shapes and/or configurations. Furthermore, although the guide member is shown releasably captured to the guide support, this is not required.

Moreover, although the device to be inserted into the body of the patient is shown having an elongated shape, it should be understood that the various aspects of the present may also be used in association with devices of other shapes and type, except where otherwise stated.

It should be understood that any measurements described herein or in the drawings are for example only and are not required.

Note that, except where otherwise stated, phrases such as, for example, “extends transversely” mean “extends in a direction that has, but is not limited to, a transverse component.” Thus, for example, “extends transversely” means “extends in a direction that is purely transversely” or “in a direction that has a transverse component in addition to an axial and/or circumferential component”, which includes but is not limited to, “substantially transversely”.

Also note that, except where otherwise stated, the term “retain” means “releasably retain” or “permanently retain”.

Also note that, except where otherwise stated, the term “to support” means to “to provide at least some support”, “to provide at least some guidance” and/or combinations thereof.

Also note that, except where otherwise stated, the phrase “a device to be introduced into a body” means “a device introduced into a body” or “a device to be introduced into a body”.

Also note that, except where otherwise stated., phrases such as, for example, “connected to” mean “connected directly to” or “connected indirectly to”.

Also note that, except where otherwise stated, terms such as, for example, “comprises”, “has”, “includes”, and all forms thereof, are considered open-ended, so as not to preclude additional elements and/or features.

Thus, while there have been shown and described various embodiments, it will be understood by those skilled in the art that the present invention is not limited to such embodiments, which have been presented by way of example only, and that various changes and modifications may be made without departing from the spirit and scope of the invention. Accordingly, the invention is limited only by the appended claims and equivalents thereto.

What is claimed is:

1. Apparatus for use in supporting a device to be introduced into a body of a patient, the apparatus comprising:
a base;
a guide member that defines a channel for receiving the device to be introduced into the body of a patient; and
a guide support being removably connected to the base and releasably capturing the guide member; wherein the guide support has at least one state in which the guide support defines a first opening, the guide member has at least one state in which the guide member defines a second opening, each of said openings having a width that is at least as wide as the channel to permit the guide support and the guide member to be separated from the device without removing the device from the body of the patient.

2. The apparatus of claim 1 wherein the base further comprises a surface with adhesive adhered thereto for removably adhering the base to the body of a patient.

3. Apparatus for use in supporting a device to be introduced into a body of a patient, the apparatus comprising:
a base having a surface with adhesive adhered thereto for removably adhering the base to the body of a patient;
a guide member that defines a channel for receiving the device to be introduced into the body of the patient; and
a guide support that is removably connected to the base and releasably captures the guide member;
wherein the guide support has at least one state in which the guide support defines a first opening and the guide member has at least one state in which the guide member defines a second opening to permit the guide member to be separated from the device without removing the device from the body of the patient.

4. Apparatus for use in supporting a device to be introduced into a body of a patient, the apparatus comprising:
a base defining a mount and having a surface with adhesive adhered thereto for removably adhering the base to the body of a patient;
a first guide releasably mountable to the mount and defining a first channel having a first configuration; and
a second guide releasably mountable to the mount and defining a second channel having a second configuration that is different than the first configuration.

5. The apparatus of claim 4 wherein the first support comprises:
a first guide that defines the first channel; and
a guide support being removably mountable to the base and releasably capturing the guide member.

6. Apparatus for use in supporting a device to be introduced into a body of a patient, the apparatus comprising:
a base;
a guide member that defines a channel for receiving an device to be introduced into a body of a patient; and
a guide support including two portions pivotably connected to one another, a first positioning of the two portions defining a first state of the guide support, a second positioning of the two portions defining a second state of the guide support, the guide support further including a catch that is engaged with the base to releasably mount the guide support thereto when the guide support is in the first state and is disengaged from the base when the guide support is in the second state.

7. The apparatus of claim 6 wherein the guide member is releasably captured in the guide support when the guide support is in the second state.

8. Apparatus for use in supporting a device to be introduced into a body of a patient, the apparatus comprising:
a base;
a guide member that defines a channel for receiving the device to be introduced into the body of a patient; and
a guide support including two portions pivotably connected to one another, a first positioning of the two portions defining a first state of the guide support, a second positioning of the two portions defining a second state of the guide support, wherein the guide member is releasably captured in the guide support when the guide support is in the second state.

9. Apparatus for use in supporting a device to be introduced into a body of a patient, the apparatus comprising:
a base for releasable attachment to the body of the patient;
a guide member defining a channel for receiving the device to be introduced into the body of a patient; and
a guide support including two portions movable relative to one another, a first positioning of the two portions defining a first state of the guide support, a second positioning of the two portions defining a second state of the guide support, wherein the guide member is releasably captured in the guide support when the guide support is in the first state and at least one of the guide support portions may be moved in a direction generally parallel to a major outer surface of the base plate to release the guide.

10. The apparatus of claim 9 wherein the guide includes two portions movable relative to one another, a first positioning of the two portions defining an open state of the guide, a second positioning of the two portions defining a closed state of the guide in which state the guide defines the channel.

11. The apparatus of claim 9 wherein the two portions of the guide are pivotably connected to one another.

12. Apparatus for use in supporting a device to be introduced into a body of a patient, the apparatus comprising:
a substantially flat, flexible base; and
a guide member that defines a channel for receiving the device to be introduced into the body of a patient and is disposed in register with a marginal portion of the base;
wherein the guide member has at least one state in which the guide support defines an opening to permit the guide member to be separated from the device without removing the device from the body of the patient.
